(19) United States
(12) Patent Application Publication
(10) Pub. No.: US 2025/0152433 A1
(43) Pub. Date: May 15, 2025

(54) ABSORBENT ARTICLE WITH FILM TOPSHEET

(71) Applicant: The Procter & Gamble Company, Cincinnati, OH (US)

(72) Inventor: Pietro CECCHETTO, Fiarfield, OH (US)

(21) Appl. No.: 18/509,726

(22) Filed: Nov. 15, 2023

(51) Int. Cl. A61F 13/537; A61F 13/15; A61F 13/511; A61F 13/513

(52) U.S. Cl. CPC A61F 13/53708; A61F 13/15203; A61F 13/51121; A61F 13/513; A61F 2013/15406

(57) ABSTRACT

An absorbent personal hygiene article including a topsheet. The topsheet includes a film layer, wherein the film layer includes a pattern of cones and valleys. The cones may project upwardly from adjacent valleys to rim edges proximate an upper side of the film layer. The cones circumscribe and define apertures through the film layer. The film layer may be disposed on a fluid management layer or an absorbent core layer.

### FIELD

The present disclosure relates to absorbent articles, and more particularly, to absorbent feminine hygiene pads having a topsheet comprising a film layer.

### BACKGROUND

Absorbent articles such as feminine hygiene pads typically include a liquid permeable, wearer-facing topsheet, a liquid impermeable, outward-facing backsheet, and an absorbent structure disposed between the topsheet and the backsheet. The topsheet and backsheet are typically bonded together about and outside the perimeter of the absorbent structure, to envelope a space in which the absorbent structure is contained.

For feminine hygiene pads, it is generally desired that the liquid permeable topsheet readily accept and facilitate movement of discharged menstrual fluid quickly therethrough down to the underlying absorbent components of the absorbent structure, to minimize spreading flow of discharged fluid across the topsheet surface, and thereby reduce a risk that fluid may escape the pad and soil the user's underwear, outer clothing, bedding, etc. It is also generally desired that the topsheet not absorb or retain fluid itself, but rather, readily pass it to underlying absorbent components, so that it will feel dry to the user after a discharge of fluid to the pad. Generally, it is desired that the topsheet have a soft, compliant, cool, cloth-like feel against the wearer's skin, and that it does not generate noise when manipulated during application or moved about against the user's body during use/wear. It is also generally desired by users that the topsheet have a level of opacity that enables it to conceal, to a feasible extent, fluid that has passed therethrough and been retained in the underlying absorbent structure.

The topsheet material also should be sufficiently mechanically robust to pass without unworkable degrees of stretchability or tearing failure, through pad manufacturing process equipment. Conservation of material usage, manufacturing efficiency and cost control are ever-present objectives.

From the perspective of engineering material(s) to be used to form a topsheet, these objectives are in conflict to varying degrees, and it is difficult to serve all of them simultaneously.

Accordingly, there remain opportunities for improvements in film topsheets to form an improved consumer/user-pleasing experience that is also cost-effective and can be manufactured in a high-speed process.

### SUMMARY

In some embodiments, an absorbent personal hygiene article may include a topsheet. The topsheet may include a film layer. The film layer may include polyethylene (PE), such that the polyethylene is a homogeneous blend of low density polyethylene (LDPE) and linear low-density polyethylene (LLDPE). The homogeneous blend may be at least 50% by weight LDPE, and LLDPE. The film layer may have a basis weight of from about 14 gsm to about 24 gsm. The film layer may include a pattern of cones and valleys. The cones project upwardly from adjacent valleys to rim edges proximate an upper side of the film layer. The cones circumscribe and define apertures through the film layer. Stated another way, the rim of the cone are opened forming an aperture. The apertures have an average largest x-y dimension (ar) of about 375 μm to about 550 μm. Further, the cones have an average height (ch) of about 300 μm to about 500 μm, and the apertures have a numerical density of about 140 to about 500 apertures per cm² x-y plane surface area of the film layer. The film layer may be disposed on at least one of a fluid management layer and an absorbent core layer.

### BRIEF DESCRIPTION OF THE DRAWINGS

FIG. 1 is a schematic, expanded vertical cross sectional view of a portion of a film layer disposed on a subsequent layer.

FIG. 2 is a schematic, expanded x-y plan view of the upper side of a portion of a film layer.

FIG. 3A is a photograph of a magnified perspective view of a portion of an example of a film layer.

FIG. 3B is a photograph of a magnified vertical section of a portion of an example of a film layer.

FIG. 3C is a photograph of a further-magnified vertical section of a portion of an example of a film layer.

FIG. 4A is a photograph of a magnified plan view of the upper side of a portion of an example of a film layer.

FIG. 4B is a photograph of a further-magnified plan view of the upper side of a portion of an example of a film layer.

FIG. 4C is a photograph of a magnified plan view of the lower side of a portion of an example of a film layer.

FIG. 4D is a photograph of a further-magnified plan view of the lower side of a portion of an example of a film layer.

FIG. 5 is a schematic representation of a process for forming a film layer.

FIG. 6 is a cross-sectional, schematic representation of a portion of the apparatus shown of FIG. 5.

FIG. 7A is a schematic representation of an exemplary tooth of the apparatus shown of FIG. 5.

FIG. 7B is a schematic representation of a portion of a roll having teeth.

FIG. 8 is a schematic representation of a process for forming a film layer.

FIG. 9 is a perspective view of a schematic representation of an absorbent article.

### DETAILED DESCRIPTION

The term “absorbent article” includes disposable articles such as feminine hygiene pads (sometimes called “sanitary napkins”), panty liners, diapers and training pants, menstrual underwear, adult incontinence pads and absorbent underwear. Such absorbent articles are designed for the absorption of body exudates, such as menstrual fluid, urine, and liquidous feces. Absorbent articles contemplated herein will typically include a liquid permeable topsheet, a liquid impermeable backsheet joined to the topsheet, and an absorbent structure disposed between the topsheet and backsheet.

The term “absorbent structure”, as used herein, refers to the component or combination of components of the absorbent article that is adapted for absorbing and retaining absorbed liquids until such time the wearer/user removes the article from wear/use.

The term “aperture”, as used herein, refers to a hole through a film and/or other web material. Apertures may be punched through the web or film so that material of the film circumscribing the aperture is disposed approximately in the same plane occupied the film prior to the formation of the aperture; or apertures through a film may be created in a manner in which at least some of the film material circumscribing the aperture is deformed and displaced in a z-direction out of an original plane occupied by the film. In the latter case, the apertures may be circumscribed by a deformation of the film material.

Herein, use of the term “cone” is not intended to mean or imply compliance of the shape of the feature referred to with the definitions of “cone” or “conical” in mathematics or geometry. Rather, it is intended as a shorthand expression to describe formations in a film that result from a deformation-based aperturing process as described herein. The formations referred to as “cones” herein may be varying and irregular in shape, as reflected in the figures.

The term “cross direction” or “CD”, as used herein with respect to a web, refers to the path that is perpendicular to the machine direction and along the plane occupied by the web.

The term “machine direction” or “MD”, as used herein with respect to a web, refers to the path that the web follows through a manufacturing process.

For an absorbent article or component thereof laid out flat along a horizontal plane, the term “z-direction” refers to a direction orthogonal to the horizontal plane. For an absorbent article or component thereof, that is applied to and/or being worn by a wearer, the “z-direction” at any particular location on the article is a direction generally normal to the curvature of the article or component at that location, as it follows or wraps about the wearer's body contours.

For a component or feature of an absorbent article, the vertical references “upper”, “lower”, “uppermost”, “lowermost”, “height,” and similar terms characterizing vertical positioning or vertical dimensions, refer to positioning or dimension of the component or feature relative other components or features and relative a wearer's body when the article is used/worn. An “upper” component or feature is disposed relatively nearer the wearer's body than a “lower” component. When the article is laid out flat on a horizontal surface with wearer-facing surface facing up, the “upper” surfaces of layer components face upwardly, and the “lower” surfaces of layer components face downwardly.

The term “predominantly”, when used with respect to a quantity of numerical count, weight, volume, or linear dimension of a component present in a structure, means that a majority of the numerical count, weight, volume structure is constituted by the component.

With respect to apertures or cones present in a film, “numerical density” refers to the number of apertures present per unit x-y plane surface area of the film on one side.

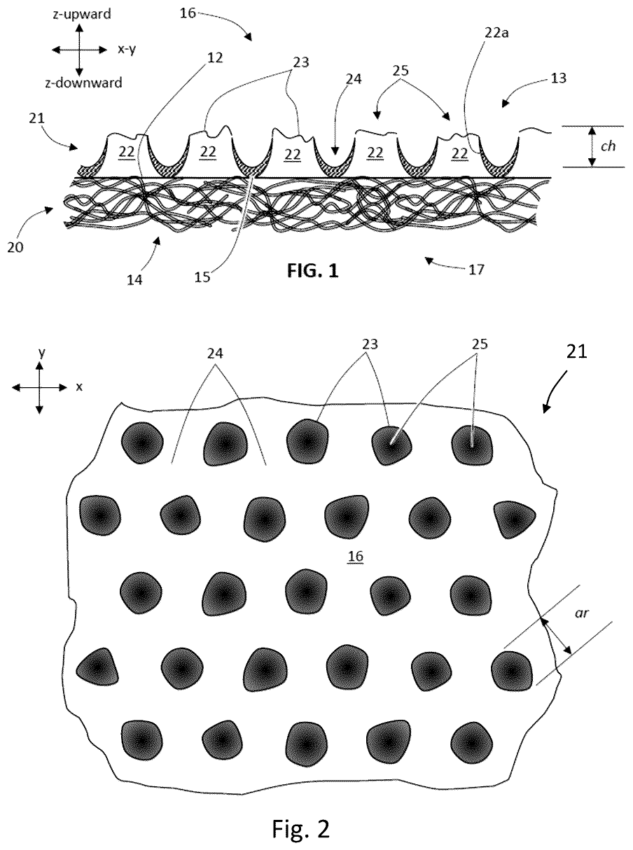

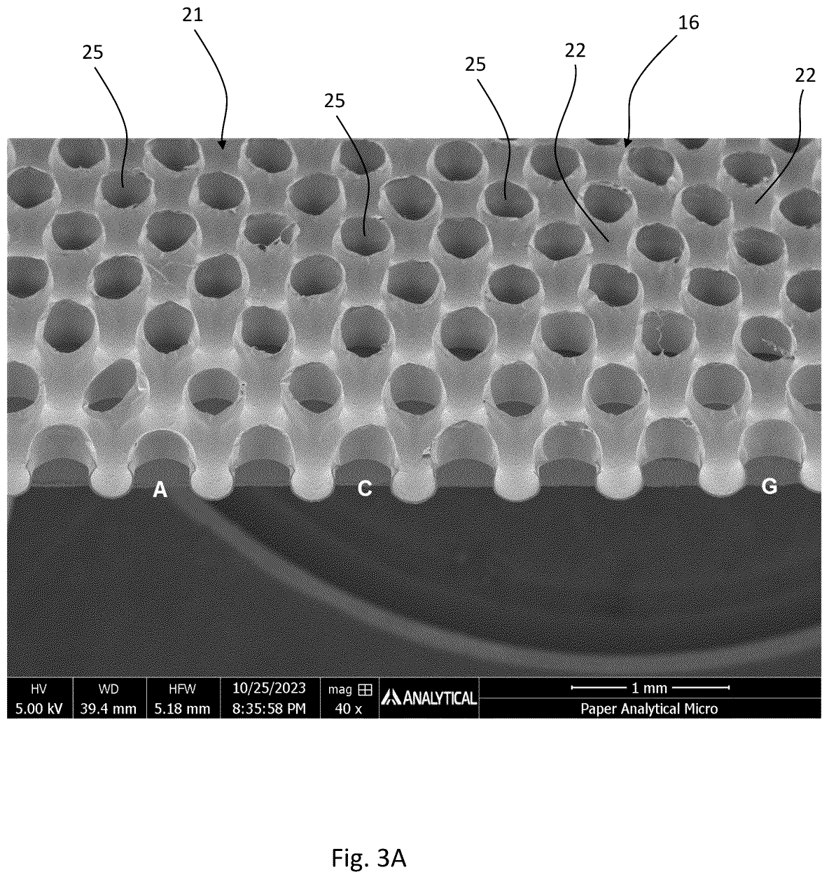

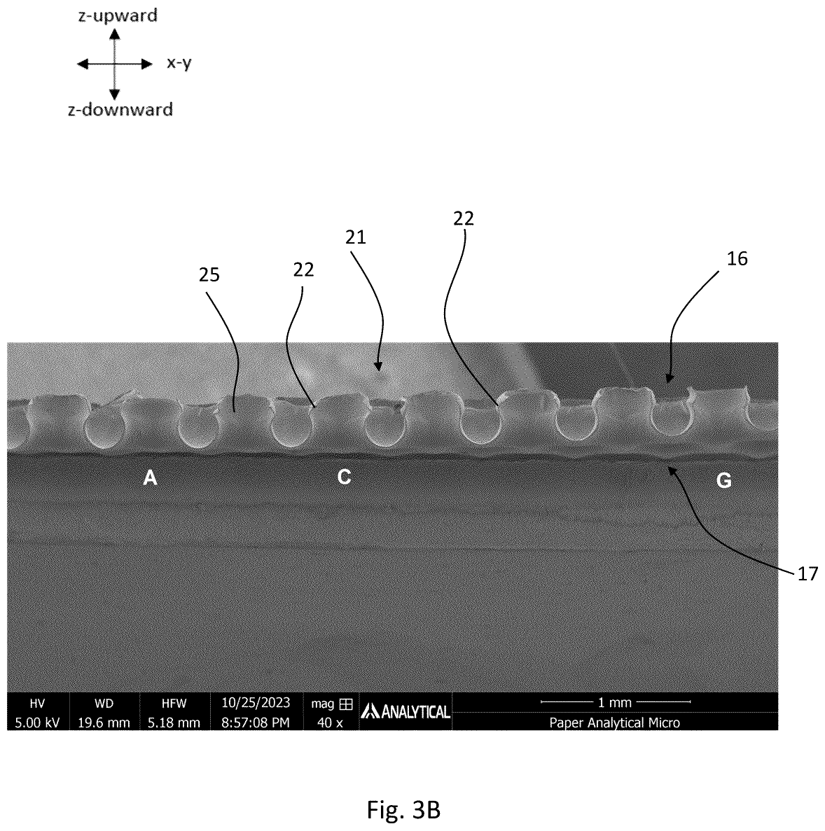

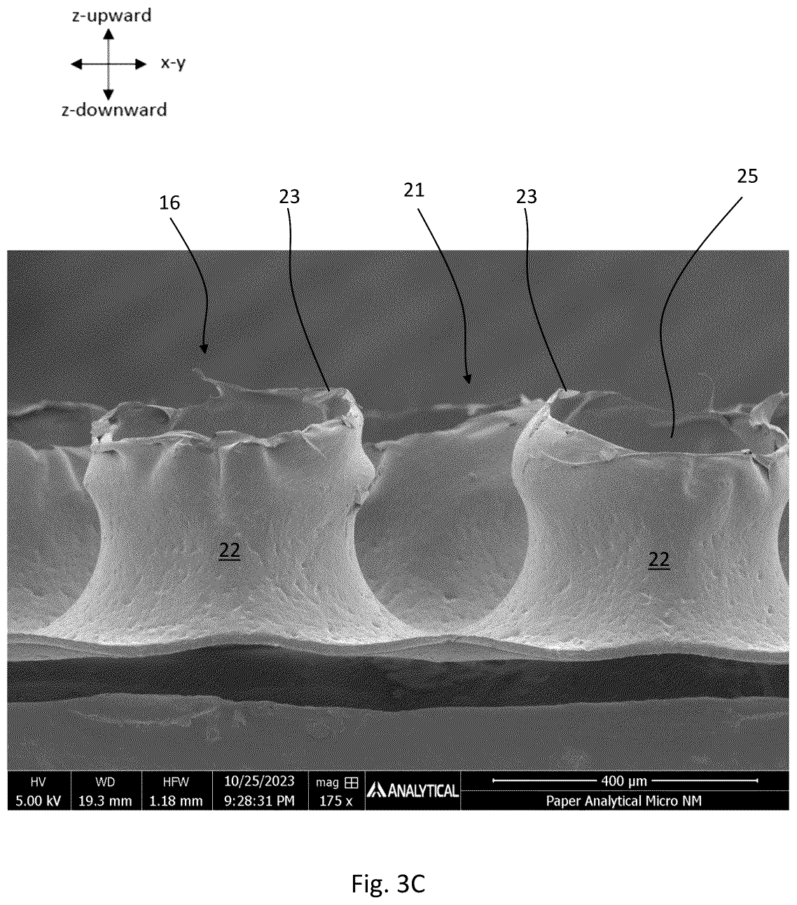

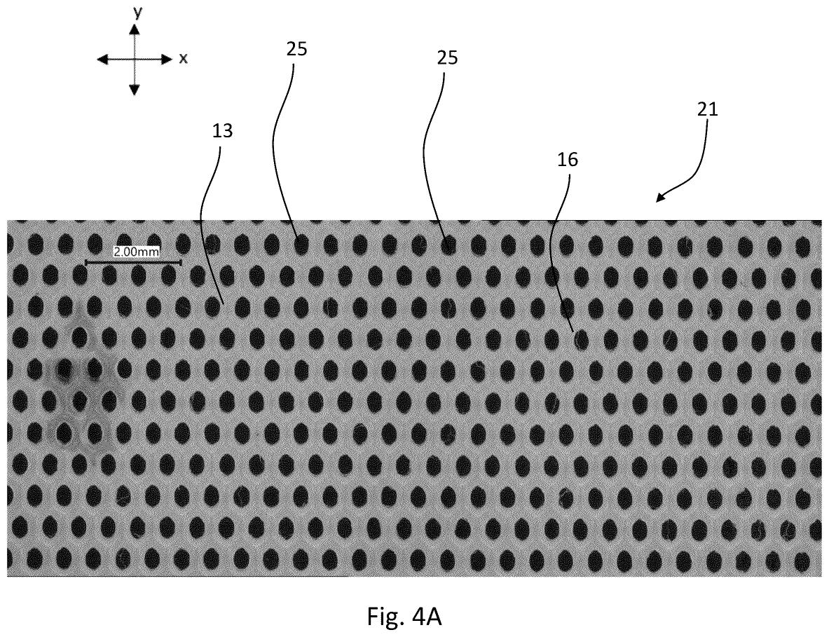

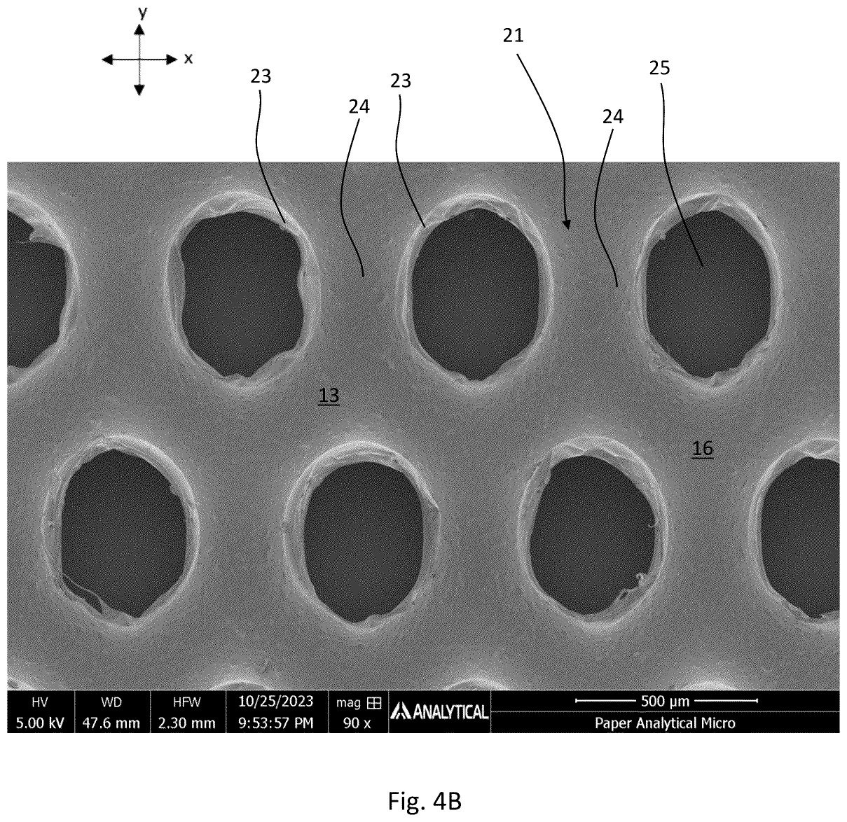

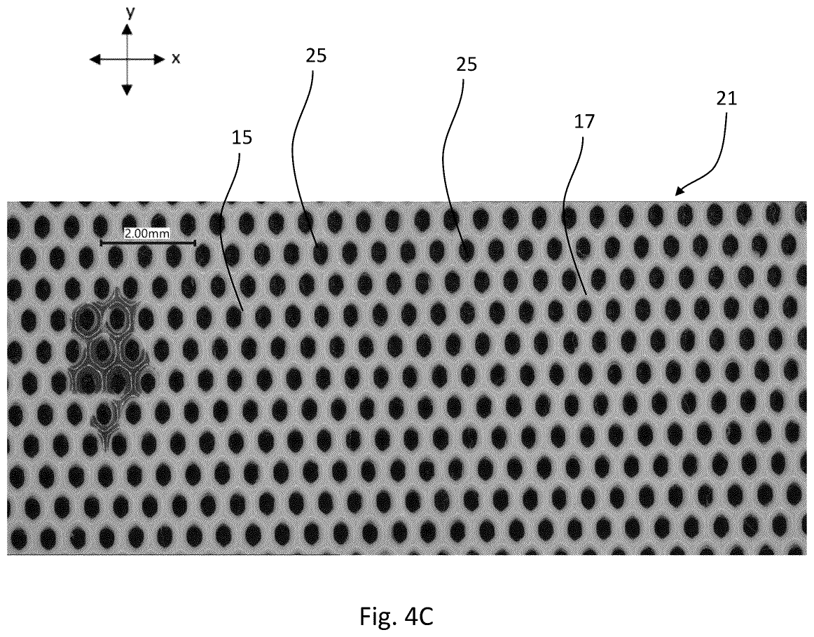

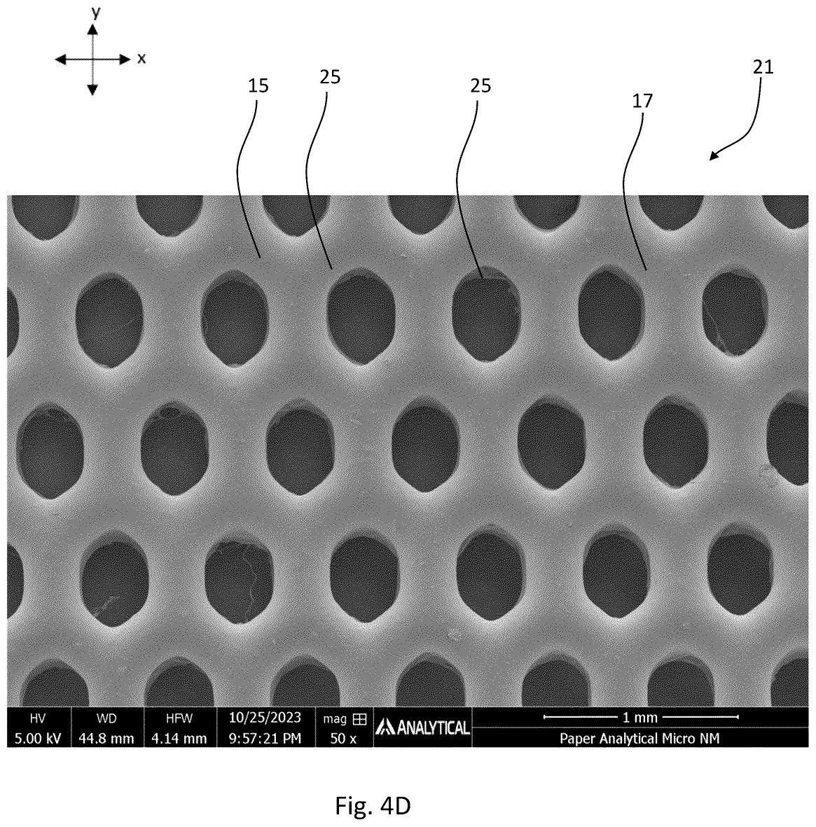

Referring to FIGS. 1 through 4D, contemplated herein is a film layer 21 comprising polymers, also referred to herein as a polymer film. The film layer 21 has an upper surface 13 and a lower surface 15. film layer 21 has a machine direction (MD) and a cross machine direction (CD) as are commonly understood in the art of web manufacturing. The layers are described herein as generally planar, having major sides lying along x-y planes and having a caliper measured along the z-direction. The film layer 21 and any additional layer(s) positioned adjacent to the film layer 21 may be joined by adhesive, thermal bonding, ultrasonic bonding or by other mechanisms. The film layer 21 and any additional layer, such as a subsequent layer 20 has an upper side 16 and a lower side 17. The term “sides” is used in the common usage of generally planar webs, such as paper and films that have two major sides when in a generally macroscopically flat condition.

The film layer 21 contemplated herein can have an opacity. To enhance opacity to a desired level, the manufacturer may blend a pigmenting, whitening, or opacifying agent (for example. TiO₂ powder) into the component resin(s) of the film layer, such as TiO₂ in a quantity of at least 1% to 10%, more preferably 5% to 9%, and even more preferably 6% to 8%, by weight of the film layer. In some examples, the film layer may have a higher weight percent quantity of whitener than typically used for films to offset the plastic appearance of the film. The selected pigmenting, whitening, or opacifying agent is preferably an inorganic pigmenting agent that will not degrade or change chemically at temperatures at which the polymers are not chemically reacting with the polymer resin(s) into which it is blended. It is generally desired to achieve as much opacity as possible, while not substantially weakening the structures of the film by overloading, and not causing excessive die wear that may be caused by abrasive inorganic pigment particles in a resin blend, rubbing against extruding dies. In some examples, a whitening agent such as TiO₂ powder may be preferred because consumers/users of absorbent articles may tend to associate white coloration with cleanliness, purity, and/or sanitariness.

### Film Layer

More specifically, referring to FIGS. 1-4D, film layer 21 may be cast, extruded or formed by other film formation processes, of one or more polymer components. In its finally formed state, the film layer 21 may include a pattern of cone formations, referred to herein as cones 22. A corresponding pattern or arrangement of valleys 24 is present between the cones 22. The cones 22 extend in an upward direction from the valleys and terminate in cone rim edges 23. The rim edges 23 and/or inner wall surfaces 22a of the cones 22 circumscribe apertures 25 through the film layer, which may be viewed along the z-direction and seen to define areas along an x-y plane.

The z-direction orientation, shapes, dimensions, geometric features, and numerical density of the cones 22 and apertures 25, as well as the composition and basis weight of film layer 21, will impact the relative degree of success or failure of the film layer at meeting the objectives for an absorbent article topsheet, as identified herein.

It has been learned to be preferable that the cones 22 project upwardly from the valleys 24 (for a topsheet, toward the wearer/user), rather than downwardly toward the underlying absorbent components, such as the absorbent core and/or the fluid management layer. This feature, combined with a suitable level of numerical density of the cones/apertures, provides for a film that largely contacts the wearer/user's skin primarily at the cone rims 23, providing for intermittent/discontinuous areas of contact between the wearer's skin and the film layer 21. For a topsheet of an absorbent article, the result is that the film layer 21, desirably, feels less like plastic film, and more like fibrous fabric, against the user/wearer's skin.

It has been learned that one or a homogeneous blend of polyethylene (PE) variants including low-density polyethylene (LDPE) and linear low-density polyethylene (LLDPE) are preferable as polymer components of film layer 21. These are preferred because they have relatively low melting temperatures, and a film constituted of one or a combination of LDPE and LLDPE contribute substantially to a soft skin feel for the upper side 16 of the film layer. Generally, LDPE tends to be softer and more pliable, contributing to soft feel, while LLDPE tends to provide more stiffness/rigidity, contributing to maintaining structural integrity of the cones. When the cones 22 are formed in the film via a process described below, the film is stretched to rupture to form apertures through the film resulting in cones, and the rim edges 23 of the cones are relatively thin from stretching prior to rupture. These rim edges contact the user's skin during use, and when the film is constituted of LDPE (supplemented by LLDPE), are quite pliable and feel soft, particularly when the cones are arranged with a size and numerical density as described herein. For this reason, such constitution of film layer 21 has been discovered to be preferable to other combinations of potential film components such as high-density polyethylene (HDPE), polypropylene, polyethylene terephthalate (PET), etc. These other polymers when included may impart a relatively undesirable level of stiffness to the film layer and the cones, which contributes to maintaining structural integrity of the cones—but this comes at the cost of soft skin feel for the user. A film layer having cones that are too stiff may actually feel scratchy to the user. It has been discovered, however, that constituting the film layer 21 only of one or a blend of LDPE and LLDPE provides for cones that sufficiently maintain structural integrity while still being pliable and impart a soft skin feel to the upper surface 13 of the film layer 21. Accordingly, the film layer 21 contemplated herein preferably is constituted of no more than 10%, more preferably no more than 5%, and even more preferably no more than 1%, HDPE, polyethylene or PET. In some embodiments, the film layer 21 may be void of HDPE.

The polymer component(s) of the film layer 21 may be naturally hydrophobic, which results in formation of a film that does not facilitate movement of aqueous fluid (such as menstrual fluid) along its surfaces and thus down through cones 22 and apertures 25, to underlying layers. However, it is desired that film layer readily accepts and facilitate passage of aqueous fluid through the apertures 25. Accordingly, film layer 21 may be surface treated with a suitable hydrophilizing agent such as a surfactant, either before or after formation of the film layer 21. Alternatively, or in addition, the polymer component(s) of the film layer 21 may have blended thereinto, prior to formation of the film, a hydrophilizing agent, suitably selected to be blended with the polymer component(s) in melt and then migrate through the film structure, and “bloom” out to the surfaces of the film.

The precursor film may be formed by any suitable process, for example, via extrusion of molten polymer constituents through a film extrusion die.

As noted, a film layer 21 with cones 22 will provide the exterior surfaces with a softer, more cloth-like texture, provide a more cloth-like appearance, and increase the overall caliper of the layer. Examples of processes to cones 22 in a film include but are not limited to vacuum formation and mechanical deformation, and any combination thereof.

The film layer may be extruded to have a basis weight of between about 14 gsm to about 24 gsm, between about 16 gsm to about 20 gsm, or about 18 gsm. If the film is manufactured to a basis weight less than about 14 gsm, it may be unacceptably vulnerable to tearing in downstream manufacturing processes, or in use of an absorbent article. If the film layer is manufactured to a basis weight greater than 25 gsm, it may be unacceptably stiff and impart unacceptable stiffness to the film layer, and reliable formation of cones with apertures and features described herein may be adversely impacted.

The film layer 21 contemplated herein includes a pattern of cones 22 formed in the film. The cones 22 may be imparted with open upper ends, terminating in rim edges 23 and having side walls 22a. The film layer 21 may be configured such that the cones 22 extend upwardly from the valleys 24. Without being bound by theory, it is believed the cones 22 extended upwardly as described and depicted herein impart perceivable softness, a relatively less plastic feel, and overall comfort to the skin when a film layer from which the cones 22 extend is the skin-facing layer in absorbent articles. The upwardly-projecting cones 22 reduce contact surface area between the film layer 21 and the wearer/user's skin. The cones 22 also provide visibly perceivable texture to the upper surface 13 of the film layer 21, reducing gloss or a shiny appearance that the film may otherwise present. Additionally, by reducing contact surface area between the wearer/user's skin and a topsheet for an absorbent article, the cones greatly reduce the potential for sticking of the topsheet to the wearer/user's skin, following a discharge of fluid to the article.

In examples in which the cones 22 have open ends and define apertures 25, the cones may be formed by applying high pressure vacuum to the inner or lower surface of a forming screen having an outside or upper forming surface of the precursor film is drawn against. Such methods of aperturing are sometimes known as “vacuum forming” and are described in greater detail in, for example, U.S. Pat. Nos. 4,463,045 and 4,552,709. Examples of alternative mechanical deformation are disclosed in U.S. Pat. Nos. 4,798,604; 4,780,352; 3,566,726; 4,634,440; WO 97/40793, and European Patent EP 525,676.

The film layer comprises cones and it is preferred that the cones 22 be created in the film to have an average height ch, from the lowest portions of their surrounding valleys 24, of about 300 μm to about 500 μm or about 400 μm, for purposes of soft, cloth-like skin feel and resistance to sticking, such as illustrated in FIG. 1. For purposes of providing for suitable softness, appearance, skin feel, resistance to sticking and fluid acquisition capability, is preferred that the apertures delineated by the cones have an average numerical density 140 to 500, more preferably 200 to 300, and even more preferably 220 to 270 apertures per cm² x-y planar surface area in z-direction plan view of the upper surface the film. For purposes of providing suitable fluid acquisition capability, it is preferred that the apertures have an average largest x-y plane dimension ar of 375 μm to 550 μm, across the open region of the aperture, between opposing circumscribing rims or side walls of the cone, such as illustrated in FIG. 2. For purposes herein, only apertures that are entirely circumscribed by and appear entirely within the outer perimeter of the sample measurement area, are counted, in determining numerical density and average largest x-y dimension (i.e., apertures that straddle the outer perimeter of the sample measurement area are not counted.) Counts of numerical density, and measurements of cone height and x-y aperture dimensions may be made using any suitable method and may be facilitated using magnified scale photography (such as SEM photography) and image analysis software. For purposes herein the sample measurement area may be any 0.25 cm², square, x-y planar area at any location of the subject topsheet film layer having cones formed therein.

### Subsequent Layers

As previously discussed, the absorbent article needs to have the ability to move fluids from the topsheet, which comprises the film layer 21, to a subsequent layer(s) 20 disposed adjacent to the film layer 21 so that the absorbent article feels dry and comfortable against the consumer's skin. As illustrated in FIG. 1, for example, the film lower surface 15 of the film layer 21 may be disposed on a surface of the subsequent layer 20. Since the topsheet of the present disclosure is a film layer, the subsequent layers are selected so that fluid adequately removed from the topsheet. In some embodiments, the film layer may be disposed directly on an absorbent core layer. In some embodiments, the film layer may be disposed on a fluid management layer that is positioned between the topsheet, which is the film layer, and an absorbent core layer. The following details provide subsequent layers that act in combination with the topsheet comprising the film layer.

In one embodiment, the film layer 21 may be disposed on a subsequent layer 20, wherein the subsequent layer 20 is a fluid management layer. It is important that the fluid management layer have adequate openness to allow for quick acquisition of fluid yet also be able to lock away liquid insults to reduce the likelihood of rewet. Examples of suitable fluid management layers are disclosed in U.S. Pat. Nos. 11,389,336; 11,717.451; and 11,389,336; and U.S. Patent Publication Nos. 2020/0315870; 2020/0315861; 2020/0315859; 2020/0315872; 2020/0315873; 2020/0315874; and 2020/0315871, which are each incorporated by reference herein. The fluid management layers of the present disclosure may comprise a plurality of carded webs. The carded webs that make up the fluid management layer may be different from one another. For example, one of the carded webs may comprise a different fiber blend than the others (heterogeneous configuration). Specifically, assuming the first carded web would be closest to the wearer-facing surface in an absorbent article, the fiber selection for a first carded web may be such that there is more openness associated with this web. A second carded web may be similarly configured. In contrast, a third carded web may be configured to collect liquid insults from the void space of the first and second carded webs and effectively distribute these liquid insults to an absorbent core layer. Alternatively, the first carded web, the second carded web and the third carded web may be configured the same, e.g., have the same fiber blend (homogeneous configuration).

The fluid management layers of the present disclosure can have a caliper factor (mm of caliper per 10 gsm) of at least about 0.13 mm, at least about 0.15, or about 0.2 mm, including any values within these ranges and any ranges created thereby. The fluid management layer 20 can have a caliper factor of between 0.13 mm to about 0.3 mm, or from about 0.14 mm to about 0.25 mm, or from about 0.15 mm to about 0.22 mm, including all values within these ranges and any ranges created thereby.

The fluid management layers of the present disclosure can have a basis weight of up to 75 grams per square meter (gsm); or a basis weight of up to 70 gsm; or a basis weight in the range of about 40 gsm to about 75 gsm; or in the range of about 50 gsm to about 70 gsm; or in the range of about 55 gsm to about 65 gsm, including any values within these ranges and any ranges created thereby. In another specific example, the fluid management layer may have a basis weight of between 40 gsm to 60 gsm.

The distance between the fibers of the fluid management layer is important so that the fluid may be pulled from the film layer to maintain dryness of the absorbent article. The fluid management layer should be designed to have a fast acquisition speed to quickly pull fluid from the film layer. It has been found that to maintain dryness of the film layer the fiber to fiber distance of the fibers adjacent to the film layer should be at least about 55 microns. Additionally, the fibers may have a diameter of at least about 20 microns to maintain adequate spacing between fibers creating void volume within the fluid management layer.

The fluid management layers of the present disclosure may comprise from about 15 percent to about 60 percent by weight, from about 20 percent to about 50 percent by weight, from about 25 percent to about 40 percent by weight, specifically including any values within these ranges and any ranges created thereby of absorbent fibers. In one specific example, fluid management layers may comprise about 30 percent by weight of absorbent fibers.

Similarly, the fluid management layers of the present disclosure may comprise from about 20 percent to about 70 percent, from about 30 percent to about 60 percent, from about 35 percent to about 50 percent by weight of resilient fibers, specifically reciting all values within these ranges and any ranges created thereby. In one specific example, fluid management layers may comprise about 40 percent by weight resilient fibers.

Additionally, the fluid management layers of the present disclosure may comprise from about 15 percent to about 60 percent, from about 20 percent to about 50 percent, or from about 25 percent to about 40 percent of stiffening fiber, specifically reciting all values within these ranges and any ranges created thereby. In one specific example, the fluid management layer 20 may comprise about 30 percent by weight stiffening fibers.

For fluid management layer, the weight percentage of stiffening fibers can be greater than or equal to the weight percentage of resilient fibers. The weight percentage of absorbent fibers can be less than the weight percentage of resilient fibers and/or stiffening fibers. In general, a higher weight percentage of absorbent fibers is considered to be beneficial in rapidly absorbent fluid insults; however, given the proximity of the absorbent fibers to the topsheet, it is beneficial for the absorbent core to dewater the absorbent fibers. Where there is a larger percentage of absorbent fibers, typically a larger core is required to dewater the absorbent fibers. This typically leads to higher costs. With this in mind, a ratio of absorbent fibers in the fluid management layers of the present disclosure to stiffening fibers by weight percentage is less than 1:1, less than 0.6:1, or less than 0.5:1, specifically reciting all values within these ranges and any ranges created thereby. Similarly, a ratio of absorbent fibers in the fluid management layers of the present disclosure to resilient fibers by weight percentage is less than 1:1, less than 0.8:1, or less than 0.7:1, specifically reciting all values within these ranges and any ranges created thereby.

Any suitable linear density of absorbent fiber may be utilized. For example, the absorbent fiber linear densities may range from about 1 dtex to about 7 dtex, about 1.4 dtex to about 6 dtex, or from about 1.7 dtex to about 5 dtex, specifically reciting all values within these ranges and any ranges created thereby. In one specific example, the absorbent fiber may comprise a linear density of about 1.7 dtex.

The absorbent fibers of the fluid management layers of the present disclosure may have any suitable shape. Some examples include trilobal, “H,” “Y,” “X,” “T,” or round. Further, the absorbent fibers can be solid, hollow, or multi-hollow. Other examples of suitable multi-lobed, absorbent fibers for utilization in the carded staple-fiber nonwovens detailed herein are disclosed in U.S. Pat. No. 6,333,108 to Wilkes et al, U.S. Pat. No. 5,634,914 to Wilkes et al., and U.S. Pat. No. 5,458,835 to Wilkes et al. The trilobal shape can improve wicking and improve masking. Suitable trilobal rayon is available from Kelheim Fibres and sold under the trade name Galaxy. While each stratum may comprise a different shape of absorbing fiber, much like mentioned above, not all carding equipment may be suited to handle such variation between/among strata. In one specific example, the fluid management layer comprises round absorbent fibers.

Any suitable absorbent fibers may be utilized. Some conventional absorbent fibers include cotton, rayon or regenerated cellulose or combinations thereof. In one example, the fluid management layer may comprise viscose cellulose fibers. The absorbent fibers may comprise staple-length fibers. The staple length of the absorbent fibers can be in the range of about 20 mm to about 100 mm, or about 30 mm to about 50 mm or about 35 mm to about 45 mm, specifically reciting all values within these ranges and any ranges created thereby.

As noted previously, in addition to absorbent fibers, fluid management layer may also comprise stiffening fibers. Stiffening fibers may be utilized to help provide structural integrity to the fluid management layer. The stiffening fibers can help increase structural integrity of the fluid management layer in a machine direction and in a cross-machine direction which can facilitate web manipulation during processing of the fluid management layer for incorporation into a disposable absorbent article. With that in mind, the constituent material of the stiffening fibers, the weight percentage of the stiffening fibers, and heat of processing should be carefully selected.

Any suitable linear density of stiffening fiber may be utilized. For example, the stiffening fiber linear density may range from about 1.7 dtex to about 12 dtex, from about 4 dtex to about 10 dtex, or from about 5 dtex to about 7 dtex, specifically reciting all values within these ranges and any ranges created thereby. In one specific example, the stiffening fibers may comprise linear density of about 5.8 dtex.

Some examples of suitable stiffening fibers include bi-component fibers comprising polyethylene and polyethylene terephthalate components or polyethylene terephthalate and co-polyethylene terephthalate components. The components of the bi-component fiber may be arranged in a core sheath arrangement, a side by side arrangement, an eccentric core sheath arrangement, a trilobal arrangement, or the like. In one specific example, the stiffening fibers may comprise bi-component fibers having polyethylene/polyethylene terephthalate components arranged in a concentric, core-sheath arrangement where the polyethylene is the sheath.

While other materials may be useful in creating a resilient structure, the stiffness of polyethylene terephthalate is needed to create a resilient structure. In contrast, the polyethylene component of the stiffening fibers can be utilized to bond to one another during heat treatment. This can help provide tensile strength to the web in both the MD and CD. Additionally, the bonding of the polyethylene component to other polyethylene components of stiffening fibers can create fixed points in the nonwoven. These fixed points can reduce the amount of fiber-to-fiber sliding which can increase the resiliency of the material.

The stiffening fibers is that the integrated nonwoven may be heat treated post fiber entanglement. The heat treatment can provide additional structural integrity to the integrated nonwoven by forming bonds between adjacent stiffening fibers. So, where there is a higher percentage of stiffening fibers, more connection points may be created. However, too many connection points can yield a much stiffer fluid management layer which may negatively impact comfort. Regarding the heat stiffening process, any suitable temperature may be utilized. And, the suitable temperature may be impacted, in part, by the constituent chemistry of the stiffening fibers as well as by the processing fluid management layer. For example, the fluid management layer may be heat stiffened at a temperature of 132 degrees Celsius.

As noted previously, the fluid management layer of the present disclosure may additionally comprise resilient fibers. The resilient fibers can help the fluid management layer maintain its permeability and compression recovery. Any suitable size fiber may be utilized. For example, the resilient fibers can have a linear density of about 1 dtex to about 12 dtex, from about 2 dtex to about 7 dtex, or from about 3 dtex to about 5 dtex, specifically reciting all values within these ranges and any ranges created thereby. In one specific example, the resilient fibers may comprise a linear density of about 4.4 dtex. In another specific example, the fluid management layer may comprise resilient fibers having variable cross sections, e.g., round and hollow spiral, and/or may comprise resilient fibers having variable dtex's.

The resilient fibers can be any suitable thermoplastic fiber, such as polypropylene (PP), polyethylene terephthalate, or other suitable thermoplastic fibers known in the art. The staple length of the resilient fibers can be in the range of about 20 mm to about 100 mm, or about 30 mm to about 50 mm or about 35 mm to about 45 mm. The thermoplastic fibers can have any suitable structure or shape. For example, the thermoplastic fibers can be round or have other shapes, such as spiral, scalloped oval, trilobal, scalloped ribbon, and so forth. Further, the PP fibers can be solid, hollow or multi-hollow. The resilient fibers may be solid and round in shape. Other suitable examples of resilient fibers include polyester/co-extruded polyester fibers. Additionally, other suitable examples of resilient fibers include bi-component fibers such as polyethylene/polypropylene, polyethylene/polyethylene terephthalate, polypropylene/polyethylene terephthalate. These bi-component fibers may be configured as a sheath and a core. The bi-component fibers may provide a cost-effective way to increase basis weight of the material while additionally enabling optimization of the pore size distribution.

The resilient fibers can be polyethylene terephthalate (PET) fibers, or other suitable non-cellulosic fibers known in the art. The PET fibers can have any suitable structure or shape.

For example, the PET fibers can be round or have other shapes, such as spiral, scalloped oval, trilobal, scalloped ribbon, hollow spiral, and so forth. Further, the PET fibers can be solid, hollow or multi-hollow. In one particular example, fibers may be fibers made of hollow/spiral PET. Optionally, the resilient fibers may be spiral-crimped or flat-crimped. The resilient fibers may have a crimp value of between about 4 and about 12 crimps per inch (cpi), or between about 4 and about 8 cpi, or between about 5 and about 7 cpi, or between about 9 and about 10 cpi. Particular non-limiting examples of resilient fibers can be obtained from Wellman, Inc. Ireland under the trade names H1311 and T5974. Other examples of suitable resilient fibers for utilization in the carded staple-fiber nonwovens detailed herein are disclosed in U.S. Pat. No. 7,767,598 to Schneider et al.

It is worth noting that the stiffening fibers and resilient fibers should be carefully selected. For example, while the constituent chemistries of the stiffening fibers and the resilient fibers may be similar, resilient fibers should be selected such that their constituent material's melting temperature is higher than that of the stiffening fibers. Otherwise, during heat treatment, resilient fibers would bond to stiffening fibers and vice versa and could create an overly rigid structure. Note that where the stiffening fibers comprise bicomponent fibers, i.e., core/sheath configuration, the resilient fibers may comprise the constituent chemistry of the core.

In some embodiments, the topsheet, film layer 21, may be disposed directly on subsequent layer 20, wherein the subsequent layer is an absorbent core layer. The absorbent core layer serves as a storage layer for bodily exudates. The configuration and construction of the absorbent core layer may vary (e.g., the absorbent core layer may have varying caliper zones, a hydrophilic gradient, a superabsorbent gradient, or lower average density and lower average basis weight acquisition zones). The size and absorbent capacity of the absorbent core layer may also be varied to accommodate a variety of wearers. However, the total absorbent capacity of the absorbent core layer should be compatible with the design loading and the intended use of the disposable absorbent article. For example, the absorbent capacity of the absorbent core layer may be selected in view of the other adjacent layers in the absorbent article.

The absorbent core layer can contain conventional absorbent materials. The absorbent core layer may include fibers that includes or consists predominately (by weight) or entirely of cellulosie fibers. In addition to conventional absorbent materials such as creped cellulose wadding, fluffed cellulose fibers, rayon fibers, wood pulp fibers also known as air-felt or pulp fibers, and textile fibers, the core often includes superabsorbent material that imbibes fluids and form hydrogels. Such materials are also known as absorbent gelling materials (AGM) and may be included in particle form. AGM is typically capable of absorbing large quantities of body fluids and retaining them under moderate pressures. In some embodiments, the absorbent core layer may be void of superabsorbent material.

In some embodiments, the absorbent core is an airlaid absorbent core including pulp fibers, or pulp fibers and absorbent gelling material. More specifically, the absorbent core layer may include a stratum of cellulosic material, such as cellulosic fibers. The cellulosic fibers may be pulp fibers. The pulp fibers may be combined into a stratum via airlaid technology. Pulp fibers may be at least one of softwood pulp, hardwood pulp, bamboo fibers, hemp fibers, and eucalyptus fibers. In some embodiments, the cellulosic fibers may be stapled and crimped fibers, which may include rayon fiber that are combined via a spunlace or needle punch process. The absorbent core layer may include a single layer or two or more stratum of cellulosic material. For example, the absorbent core may include at least one stratum with pulp fibers and at least one stratum with rayon fibers.

The absorbent core layer may have a basis weight of at least about 75 gsm, or at least about 100 gsm, or at least about 140 gsm, or at least about 144 gsm, or at least about 150 gsm, or at least about 160 gsm, or at least about 165 gsm, or at least about 170 gsm, or from about 75 gsm to about 230 gsm or from about 90 gsm to about 185 gsm, reciting for said range every 5 gsm increment therein.

The absorbent core layer of the present disclosure may comprise any suitable shape including but not limited to an oval, a disco-rectangle, a rectangle, an asymmetric shape, and an hourglass. For example, in some forms of the present invention, the absorbent core may comprise a contoured shape, e.g., narrower in the intermediate region than in the end regions. As yet another example, the absorbent core may comprise a tapered shape having a wider portion in one end region of the pad which tapers to a narrower end region in the other end region of the pad. The absorbent core layer may comprise varying stiffness in the MD and CD.

To maintain dryness of the film layer the fiber to fiber distance of the fibers adjacent to the film layer should be at least about 55 microns. Additionally, the fibers may have a diameter of at least about 20 microns to maintain adequate spacing between fibers creating void volume within the absorbent core layer.

The film layer 21 may be bonded directly to the absorbent core layer or the fluid management layer. The film layer may be bonded by mechanical and/or chemical bonding methods, such by pressure bonding, ultrasonic bonding, and/or adhesive bonding.

### Apparatus and Method for Manufacturing

As previously discussed, the cones 22, formed by aperturing the film will increase the fluid handling properties of the film layer and provide the film layer with a more cloth-like, fiber-like appearance. Cones 22 in a film layer can be formed using any processes known in the art. Examples of such processes include but are not limited to the following: vacuum forming, hydroforming, hydro-cutting, mechanical deformation, ultrasonics, slitting, ring-rolling, and any combination thereof.

Methods for vacuum formation, hydroforming, mechanical deformation, and ultrasonics are described above. With respect to ultrasonics, additional methods are disclosed in U.S. Pat. Nos. 5,269,981 and 5,269,981. Methods of hydro-cutting are disclosed in U.S. Pat. No. 5,567,736. Suitable slitting methods are disclosed in PCT Publication WO 97/31601. In one embodiment, the cones may be formed by a mechanical deformation process. The mechanical deformation process can be carried out on any suitable apparatus that may comprise any suitable type(s) of forming structure. Suitable types of forming structures include, but are not limited to a pair of rolls that define a nip therebetween; pairs of plates; belts, etc. Using an apparatus with rolls can be beneficial in the case of continuous processes, particularly those in which the speed of the process is of interest. Although the apparatuses will be described herein for convenience primarily in terms of rolls, it should be understood that the description will be applicable to forming structures that have any other suitable configurations.

The rolls for a vacuum and/or mechanical deformation process forming the cones described herein are typically generally cylindrical. The term “generally cylindrical”, as used herein, encompasses rolls that are not only perfectly cylindrical, but also cylindrical rolls that may have elements on their surface. The term “generally cylindrical” also includes rolls that may have a step-down in diameter, such as on the surface of the roll near the ends of the roll. The rolls are also typically rigid (that is, substantially non-deformable). The term “substantially non-deformable”, as used herein, refers to rolls having surfaces (and any elements thereon) that typically do not deform or compress under the conditions used in carrying out the processes described herein. The rolls can be made from any suitable materials including, but not limited to steel, aluminum, or rigid plastic or rubber, such as for the female roll. The steel may be made of corrosion resistant and wear resistant steel, such as stainless steel. The rolls may or may not be heated. If heated, consideration of thermal expansion effects must be accommodated according to well known practices to one skilled in the art of thermo-mechanical processes.

The rolls may have elements on their surface (or surface configuration) to form the cones on a film. The surface of the individual rolls may be provided with forming elements comprising: male elements such as discrete projections and teeth; female elements such as recesses such as discrete voids in the surface of the rolls; or any suitable combination thereof. The female elements may have a bottom surface (which may be referred to as depressions, or cavities), or they may be in the form of apertures (through holes in the surface of the rolls). In some embodiments, the forming elements on the members (such as the rolls) of the forming apparatus may comprise the same general type (that is, the opposing components may both have forming elements thereon, or combinations of forming and mating elements). The forming elements may have any suitable configuration.

A film layer comprising cones according to the present disclosure can be produced from a three dimensional precursor film having a plurality of discrete extended elements by forming a plurality of discrete extended elements on the precursor film. A film comprising cones according to the present disclosure can also be produced from a generally planar, two dimensional precursor film by forming a plurality of discrete extended elements on the precursor film and forming apertures on the precursor film. For the film disclosed herein, the rolls are used to form a plurality of discrete extended elements to form the micro cone apertures.

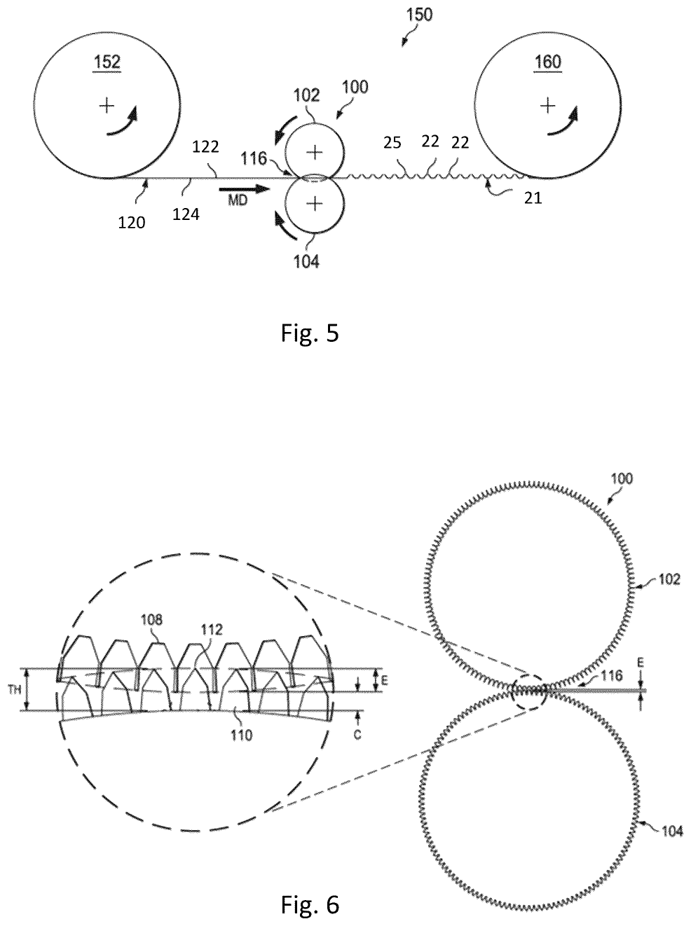

One process 150 producing a film layer including cones is shown schematically in FIG. 5. Precursor film 120 is moved in the machine direction MD to forming apparatus 100 where cones 22 are formed. Precursor film 120 can be supplied from a supply roll 152 (or supply rolls, as needed for multiple layers) or any other supply means, such as festooned webs, as is known in the art. In one embodiment, precursor film 120 can be supplied directly from a web/film making apparatus, such as a polymer film extruder. Subsequent to formation, the film layer comprising cones 22 can be taken up on a supply roll 160 for storage and further processing as a component in other products. Alternatively, the film layer 21 comprising cones 22 can be conveyed directly to further post processing, including a converting operation for incorporation into a finished product, such as a disposable absorbent product.

As illustrated in FIG. 5, a film layer can be formed from a three dimensional or flat precursor film 120 a first surface 122 and a second surface 124. First surface 122 corresponds to a first side of precursor film 120, as well as a first side of a film layer 21 comprising cones 22. Second surface 124 corresponds to a second side of precursor film 120, as well as a second side of a film layer 21 comprising cones. In general, the term “side” is used herein in the common usage of the term to describe the two major surfaces of generally two-dimensional webs, such as films. Of course, in a composite or laminate structure, the first surface 122 is the first side of one of the outermost layers or plies, and the second surface 124 is the second side of the other outermost layer or ply.

Supply roll 152 rotates in the direction indicated by the arrow in FIG. 5 as precursor film 120 is moved in the machine direction MD by means known in the art, including over or around any of various idler rollers, tension-control rollers, and the like (all of which are not shown) to the nip 116 formed by a pair of counter-rotating, intermeshing rolls 102 and 104. The pair of intermeshing rolls 102 and 104 operate to form cones including apertures therein in the film.

As illustrated in FIG. 5, the portion of forming apparatus 100 comprises a pair of intermeshing rolls 102 and 104 rotating in opposite directions. Forming apparatus 100 can be designed such that precursor film 120 remains on roll 104 through a certain angle of rotation. While FIG. 5 illustrates a precursor film 120 going straight into and the film layer 21 comprising cones coming straight out of nip 116, precursor film 120 or the film layer 21 comprising cones can be partially wrapped on either of rolls 102 or 104 through a predetermined angle of rotation prior to (for precursor film 120) or after (for the film web or layer comprising cones) nip 116. For example, after exiting nip 116, the film layer 21 can be directed to be wrapped on roll 104 through a predetermined angle of rotation such that the cones remain resting over, and “fitted” onto, teeth of roll 104.

Rollers 102 and 104 may be made of steel, aluminum, an alloy metal, and rigid plastic or rigid rubber. For example, the roll including female elements or recesses may be a rigid rubber roll. Rollers 102 and 104 may be made of a corrosion resistant and wear resistant metal.

Roll 102 may comprise one or more discrete recesses or voids into which one or more of teeth of roll 104 mesh. The recess 108 may have the same shape as a base of the teeth and slightly larger dimensions on all edges and side than the base of the teeth. The depth of the recess may be deeper than a height of the teeth. The recess may or may not be tapered. In the case, the spacing of apertures is limited by the spacing of the recesses on roll 102. Thus, it would not be possible to form apertures in the film that have a smaller center-to-center spacing than the center-to-center spacing of the recesses on roll 102. A center-to-center distance of two adjacent apertures is a measure between centers of two adjacent apertures. A point where major axis and a minor axis of an aperture cross each other is determined as a center of the aperture.

Roll 104 comprises a plurality of rows of circumferentially-spaced teeth that extend in spaced relationship about at least a portion of roll 104. Teeth are arranged in a staggered pattern. Teeth 110 extend radially outwardly from the surface of the roll 102 to engage recesses 108 of roll 102, which is shown in greater detail in the cross sectional representation of FIG. 6, discussed below. Both or either of rolls 102 and 104 can be heated by means known in the art such as by incorporating hot oil filled rollers or electrically-heated rollers. Alternatively. both or either of the rolls may be heated by surface convection or by surface radiation.

Teeth 110 can be joined to roller 104. The term “joined to” encompasses configurations in which an element is secured to another element at selected locations, as well as configurations in which an element is completely secured to another element across the entire surface of one of the elements. The term “joined to” includes any known manner in which elements can be secured including, but not limited to mechanical entanglement. Teeth can be attached to, such as by welding, compression fit, or otherwise joined. However, “joined to” also includes integral attachment, as is the case for teeth machined by removing excess material from roller 104. The location at which teeth 110 are joined to roller 104 is the base. At any cross-sectional location parallel to the base each tooth can have a non-round cross-sectional area. In an alternate embodiment the teeth may comprise pins that are rectangular or other shapes depending on the corresponding aperture shape desired. For example, the teeth may be oval shapes.

FIG. 6 shows in cross section a portion of the intermeshing rolls 102 and 104 including representative teeth 110. As shown, teeth 110 have tooth height TH, depth of engagement E, and gap clearance C. A tooth height TH may range from about 0.4 mm to about 2 mm. Depth of engagement E is a measure of the level of engaging rolls 102 and 104 and is measured from a top surface of the roll 102 to tip 102 of tooth 110 of the roll 104. Gap clearance Cis a distance between a top surface of the roll 102 and a bottom surface of the roll 104 when rolls 102 and 104 are in maximum engagement. Gap clearance is set to provide the optimal gap to form the micro cone/aperture structures, the gap tends to be as narrow as possible but wider than the film thickness of 20 microns.

The size and shape of the tooth tip 112 may be specified via the tip radius TR. The depth of engagement E, tooth height TH, clearance C, and tip radius TR can be varied as desired depending on the desired characteristics of apertures in the film layer of the present disclosure. It is also contemplated that the size, shape, orientation and spacing of the teeth 110 can be varied about the circumference and width of roll 104 to provide for varied film properties and characteristics.

Additionally, substances such as lotions, ink, surfactants, and the like can be sprayed, coated, slot coated, extruded, or otherwise applied to the precursor film or the film layer before or after entering nip 116. Any processes known in the art for such application of treatments can be utilized.

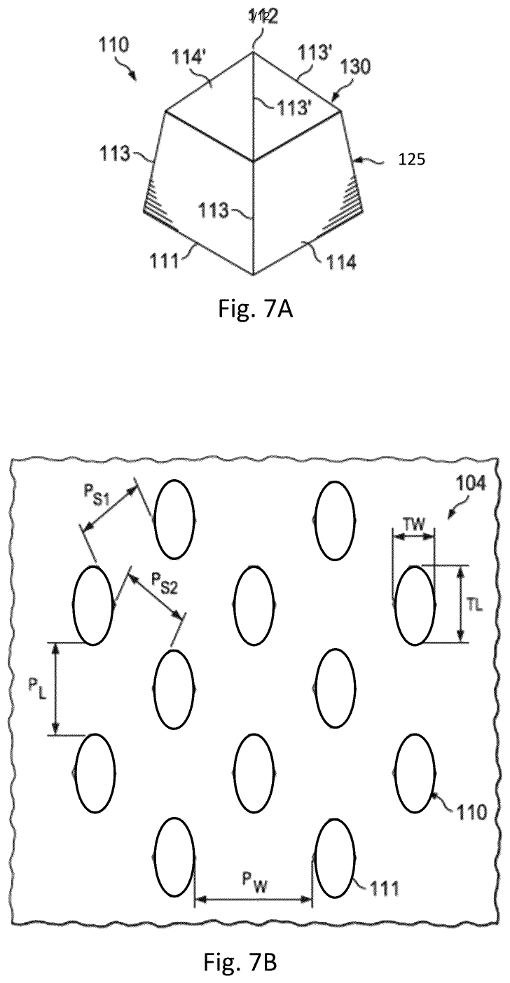

A perspective view of an exemplary configuration for teeth 100 is shown in FIG. 7A. As shown in FIG. 7A, each tooth 110 has a base 111, a tooth tip 112, edges 113, and sides 114. Teeth 110 can have a base in a generally polygonal shape. Teeth 110 may be elongated in one dimension or two dimensions, having generally non-round, elongated cross-sectional configurations. For example, at their base 111, the cross section of teeth 110 can have a tooth cross-sectional length TL and a tooth cross-sectional width TW exhibiting a tooth aspect ratio AR of TL/TW of not greater 2, or not greater than 1.5, or about 1. In some embodiments, each of the teeth has a quadrilateral shape base. The teeth 110 are tapered from a base to a tip in a way that a degree of taper is not constant along the height of the teeth. The tooth 110 may comprise a proximal part joined to a member of a forming apparatus, and a distal part 130 directly adjacent to the proximal part and tapering to a tooth tip 112. The tooth 110 may comprise a proximal part 125, a distal part 130, and a middle part between the proximal part 125 and the distal part 130. The proximal part and the distal part may have different degree of taper from each other. In one embodiment, the distal part 130 has a higher degree of taper than the proximal part 125. In another embodiment, at least one of the proximal part 125 and the distal part 130 has a constant degree of taper. The proximal part is generally a fructum shape tapering from a polygonal-shape base to a point. A proximal part 125 may have four sides 114, each side being generally (isosceles) rectangular. The vertex of two sides makes up an edge. The vertices of edges 113 can be relatively sharp or can be machined to have a rounded radius of curvature. As illustrated in FIG. 7A, a distal part 130 may have a generally pyramid shape having at least four sides 114′, each side being substantially triangular and tapering from the bottom of the distal part to a tip of the tooth. The vertex of two sides of the distal part 130 makes up an edge. The vertices of edges 113′ may be relatively sharp or may be machined to have a rounded radius of curvature. The tooth tip 112 may be generally pointed, blunt pointed, or otherwise shaped so as to stretch and/or puncture the precursor film 120. The outermost tips 112 of the teeth have sides that may be rounded to avoid cuts or tears in the precursor material. It is to be appreciated that the teeth may have any shape desired that forms cones have the properties discussed herein. For example, the teeth may be round, pin-like shapes that are generally round in cross section.

In another embodiment, other tooth shapes can be utilized to make cones, such as circular, oval, and/or pyramid shaped. For example, the generally pyramidal shape of distal part 130 may be truncated so as to remove the pointedness of tips 112 and a flattened region is produced at the distal end of tooth 110. The flattened region can also be elongated, that is, having a length dimension greater than a width dimension and an aspect ratio AR corresponding to the aspect ratio of tooth 110. In one embodiment, a flattened region can transition to sides 114 at generally sharp vertices, or the transition can be at a radius of curvature, providing for a smooth, rounded, flattened tooth tip. Without being bound by theory, it is believed that having relatively sharp tips on teeth 110 permits the teeth 110 to punch through precursor web 120 “cleanly”, that is, locally and distinctly, so that the resulting film layer comprising cones can be described as being predominantly “apertured” rather than predominantly “embossed”. In one embodiment, puncture of precursor web 120 is clean with little deformation of web 120, such that the resulting web is a substantially two-dimensional apertured web.

Another exemplary configuration for teeth 110 is shown in FIG. 7B. As illustrated in FIG. 7B, the teeth 110 may be oval shaped. The teeth 110 having a cross sectional length TL and a cross sectional width TW are arranged in a staggered pattern to have a tooth-to-tooth spacing P_{L} between two adjacent teeth along the cross-sectional length dimension, a tooth-to-tooth spacing P_{W} between two adjacent teeth along the cross-sectional width dimension, and a tooth-to-tooth spacing Ps between two adjacent teeth along a line that is not parallel either to the cross-sectional length dimension or to the cross-sectional width dimension. The teeth 110 may have different lengths of tooth-to-tooth spacing P_{S}. P_{S1} and P_{S2}, depending on teeth configuration. In some embodiments, a tooth-to-tooth spacing Ps between two adjacent teeth along a line that is not parallel either to the cross-sectional length dimension or to the cross-sectional width dimension not greater than or equal to about 250 microns. In some embodiments, at least one of the tooth-to-tooth spacing P_{L}. and P_{W} is greater than about 250 microns. It is to be appreciated that the tooth-to-tooth spacings P_{L}, P_{W} and/or Ps, tooth cross sectional length TL, and tooth cross sectional width TW may each be varied independently.

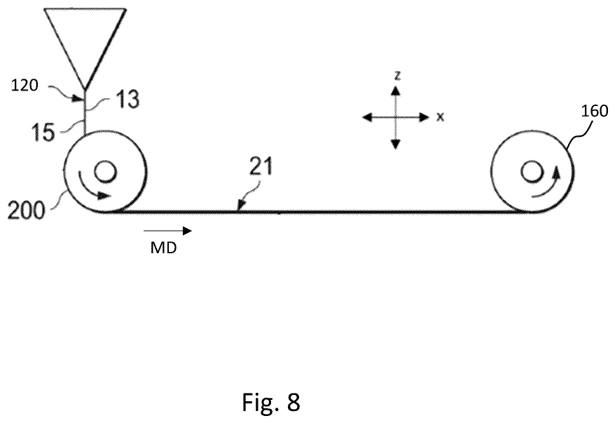

Referring to FIG. 8, an example of a process for producing a polymer film web or layer 21 by forming a pattern of cones via a cone forming unit or forming roll 200. Cone forming unit 200 may be a vacuum forming roller with an outer cylindrical forming surface formed with a pattern of holes therein, reflecting the pattern, shape, size and numerical density of cones desired to be imparted to the film. Generally, the configuration of the pattern of holes on the forming roller may be adjusted to impart the desired features to the cones, along with other process variables including film composition, basis weight and temperature, throughput speed, etc. As noted above, the forming roller may be controllably cooled for additional control over the process. Vacuum may be drawn from inside the roller, creating a pressure differential between the inside and outside of the roller, which draws the film into the holes to form cones that approximately reflect the shapes of the holes and walls thereof, which with suitable control can be caused to rupture and thereby form apertures through the film. As illustrated in FIG. 8, a precursor film 120 including an upper film surface 13 and a lower film surface 15 is advanced in a machine direction MD toward a forming unit or forming roll 200. The film 120 is disposed on the forming unit 200. Vacuum is applied to the forming unit 200 such that portions of the film are pulled into the pattern of holes or cavities, which may be of any shape, to form cones in the film. The film continues to advance about the axis of the forming roll as the cones are formed. The film layer 21 including the cones 22 with formed apertures 23 and the formed cone valleys 24 advances from the forming roll in the machine direction MD. Optionally, the film layer 21 may be wound about a supply roll 160 or advanced to additional process.

The cones 22 in film layer 21 can be formed using any processes known in the art including but not limited to those described herein.

### Application of Film Layer

The film layer contemplated herein can be used to make components of disposable absorbent articles such as but not limited to incontinence pads and pants, diapers, training pants, feminine hygiene pads/sanitary napkins and disposable menstrual pants. The film layer contemplated herein is preferably used as a topsheet for an absorbent article. The upper surface of the film layer having a pattern of upwardly-projecting cones is in contact with the skin.

An absorbent article according to the present invention comprises a topsheet and a backsheet joined to the topsheet, wherein the topsheet comprises the film layer including cones according to the present invention. The absorbent article may further comprise an absorbent core between the topsheet and the backsheet. The absorbent article may be produced industrially by any suitable means. The different layers may be assembled using standard conventional processes such as embossing, thermal bonding, or gluing or combination of both.

Any conventional backsheet materials commonly used for absorbent articles may be used as backsheet. The backsheet may be impervious to fluids. In some embodiments, the backsheet may be impervious to malodorous gases generated by absorbed bodily discharges, so that the malodors do not escape. The backsheet may or may not be breathable.

It may be desirable that the article further include an absorbent structure disposed between the topsheet and the backsheet. As used herein, the term “absorbent structure” refers to a material or combination of materials suitable for absorbing, distributing, and storing fluids such as urine, blood, menses, and other body exudates. Any conventional materials for absorbent structure suitable for absorbent articles may be used as absorbent structure.

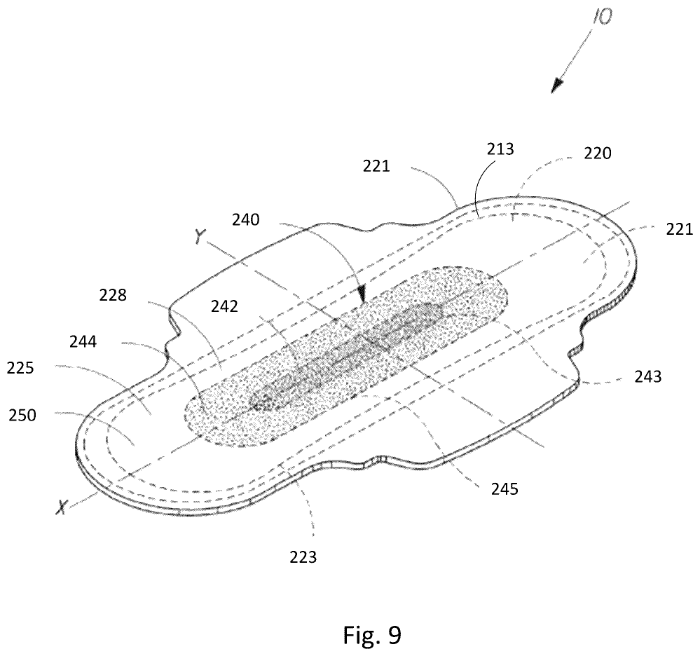

The absorbent article may include additional components and/or layers, such as a fluid management layer, and a secondary backsheet material. The absorbent article may also include indicia that communicates features of the absorbent article to the consumer, such as absorbency, orientation, and use. For example, an absorbent article may include indicia highlighting the areas of increased absorbency for the user or of channel(s) or embossment(s) placed for fluid handling. Examples of absorbent articles are described in U.S. Pat. Nos. 9,693,913 and 8,039,685, which are incorporated herein by reference. FIG. 9 illustrated a perspective view of an absorbent article 10, such as an incontinence pad. The absorbent article 10 herein has an upper surface 213, a lower surface (not seen) and a periphery 212 comprising a topsheet 225 having a bottom surface (not shown) and a top surface 228 positioned opposite to the bottom surface. The top surface 228 faces upwardly towards the upper surface 213 of the absorbent article 10. The absorbent article 10 further comprises a backsheet having a garment facing surface (not shown) and a user facing surface positioned oppositely to the garment facing surface, the backsheet being joined to the topsheet 225.

The absorbent article 10 also comprises an absorbent core 220 having a top surface 221 and a bottom surface (not shown) that is positioned opposite to the top surface 221. The absorbent core 220 may be positioned between the topsheet 225 and the backsheet. The absorbent core 220 includes a periphery 223 that defines a core shape. The shape of the absorbent core 220 may be generally rectangular, circular, oval, elliptical, or the like. The absorbent core may be made from various absorbent materials such as multiple plies of creped cellulose wadding, fluffed cellulose fibers, wood pulp fibers also known as airfelt, textile fibers, a blend of fibers, a mass or batt of fibers, airlaid webs of fibers, a web of polymeric fibers, and a blend of polymeric fibers.

To prevent absorbed bodily exudates from contacting the wearer's garments, absorbent article 10 can have a liquid impermeable backsheet. Backsheet can comprise any of the materials known in the art for backsheets, such as polymer films and film/nonwoven laminates. To provide a degree of softness and vapor permeability for the garment-facing side of absorbent article 10, backsheet can be a vapor permeable outer layer on the garment-facing side of the absorbent article 10. The backsheet can be formed from any vapor permeable material known in the art. Backsheet can comprise a microporous film, an apertured formed film, or other polymer film that is vapor permeable, or rendered to be vapor permeable, as is known in the art. One suitable material is a soft, smooth, compliant, vapor pervious material, such as a nonwoven web that is hydrophobic or rendered hydrophobic to be substantially liquid impermeable. A nonwoven web provides for softness and conformability for comfort, and can be low noise producing so that movement does not cause unwanted sound.

To provide for softness next to the body, absorbent article 10 can have a body-facing layer, referred to herein as topsheet 225. Topsheet 225 can be formed from any soft, smooth, compliant, porous material which is comfortable against human skin and through which fluids such as urine or vaginal discharges can pass. Topsheet 225 can comprise fibrous nonwoven webs and can comprise fibers as are known in the art, including bicomponent and/or shaped fibers. Topsheet 225 can also be a liquid permeable polymer film, such as an apertured film, or an apertured formed film as discussed herein.

At least one, and preferably both, of topsheet and backsheet define a shape, the edge of which defines an outer periphery of the incontinence pad 10. In a preferred embodiment, both topsheet and backsheet define the incontinence pad 10 outer periphery. The two layers can be die cut, as is known in the art, for example, after combining all the components into the structure of the incontinence pad 10 as described herein. However, the shape of either topsheet or backsheet can be independently defined.

Interposed between the absorbent core and topsheet can be at least one fluid management layer as previously discussed. The fluid management layer can aid in rapid acquisition and/or distribution of fluid and is preferably in fluid communication with the absorbent core. In one embodiment, the fluid management layer does not completely cover the absorbent core 220, but it can extend laterally to core periphery. In one embodiment, topsheet, fluid management layer, or the absorbent core can be layered structures, the layers facilitating fluid transport by differences in fluid transport properties, such as capillary pressure.

As is typical for sanitary napkins and the like, the incontinence pad 10 of the present invention can have panty fastening adhesive disposed on the garment-facing side of backsheet. Panty fastening adhesive can be any of known adhesives used in the art for this purpose, and can be covered prior to use by a release paper, as is well known in the art.

In some embodiments, such as illustrated in FIG. 9, the absorbent article 10 has at least two portions, i.e., a colored portion 240 and a non-colored portion 250. The colored portion 240 and the non-colored portion 250 are viewable from the viewing surface 228 of the topsheet 225. The colored portion 240 has at least two shades, a first shade 243 and a second shade 245.

### Test Methods

### Fiber Decitex (Dtex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%+2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area ax for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10000 m \times {a}_{k} \times {ρ}_{k} \times 1 {0}^{- 6}$$

where dₖ is in units of grams (per calculated 10,000 meter length), ax is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

### Basis Weight

The basis weight of a test sample is the mass (in grams) per unit area (in square meters) of a single layer of material and is measured in accordance with compendial method WSP 130.1. The mass of the test sample is cut to a known area, and the mass of the sample is determined using an analytical balance accurate to 0.0001 grams. All measurements are performed in a laboratory maintained at 23° C.±2 C.° and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen must be as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2 C.° and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm; however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously is the caliper per 10 gsm of basis weight of the sample. So, the equation is caliper/(basis weight/10).

### Fiber-Fiber Distance Measurement

Z-direction distances between individual fibers in a nonwoven layer in a laminate sample having a film layer and a nonwoven layer is measured using micro-CT fiber-to-fiber distance measurement based on analysis of a 3D x-ray image of a sample obtained on a micro-CT instrument having a cone beam microtomograph with a shielded cabinet such as Scanco μCT 50 (Scanco Medical AG, Switzerland) and equivalents. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. Multiple individual projection images of the sample, generated as it is rotated, are collected and then reconstructed into a single 3D image. The instrument is interfaced with a computer running software to control the image acquisition and reconstruction of the raw data into a 3D image. The 3D image is then analyzed using image analysis software such as MATLAB (The Mathworks, Inc., MA, USA) and Avizo Lite (Visualization Sciences Group/FEI Company, MA, USA) and equivalents to identify and segment out the film layer from the nonwoven layer, and measure Z-direction distances between individual fibers in the nonwoven portion of the laminate sample.

### Sample Preparation:

To obtain a sample for measurement, lay a film-nonwoven laminate out flat and die cut a circular piece with a diameter of 7 mm. If the laminate is a component of an absorbent article, tape the absorbent article to a rigid flat surface in a planar configuration, and carefully separate the laminate from the other components of the absorbent article. A scalpel and/or cryogenic spray such as Cyto-Freeze (Control Company, TX, USA) can be used to remove the laminate from the other components of the absorbent article, if necessary, to avoid extension of the laminate. Once the laminate has been removed from the article, proceed with die cutting the sample as described above.

A sample may be cut from any location containing the laminate to be analyzed. When selecting a location for sampling, care should be taken to avoid embossed regions, if any, in the absorbent article where the laminate may have been crushed and/or compressed during the article making process, as well as any folds, wrinkles or tears.

### Image Acquisition:

The micro-CT instrument is set up and calibrated according to the manufacturer's specifications. The sample is placed into an appropriate holder, between two rings of a low density material, such as foam, which have an inner diameter of at least 4 mm. This allows the central region of the sample to lay horizontal and be scanned without having any other materials directly adjacent to its upper and lower surfaces. Analysis is performed within this central region. A single 3D dataset of contiguous 3 μm isotropic voxels is collected. The 3D dataset is centered on the central analysis region, having dimensions of 7 mm on each side in the XY-plane and a sufficient number of slices to fully include the Z-direction of the sample. Images are acquired with the source at 45 kVp and 88 μA with no additional low energy filter. Current and voltage settings may be optimized to produce the maximum contrast in the projection data with sufficient x-ray penetration through the sample, but once optimized held constant for all substantially similar samples. A total of 3200 projection images are obtained with an integration time of 1000 ms and 3 averages. The projection images are reconstructed using acquisition and reconstruction software accompanies the instrument into a 3D dataset having an isotropic spatial resolution of 3 μm, and saved in 16-bit RAW format to preserve the full detector output signal for analysis.

### Image Processing:

The 3D dataset is loaded into the image analysis software, and trimmed to a rectangular prism 3D image of the analysis region by removing the surrounding holder and the low density mounting material from the 3D dataset. Trimming is performed such that the maximum amount of the sample in the analysis region is retained in the 3D image, and the empty space above and below the sample is minimized. The trimmed 3D image is scaled from 16-bit to 8-bit for the purpose of convenience in data analysis, and thresholded using Otsu's method which calculates the threshold level that minimizes the weighted intra-class variance, to separate and remove the background signal due to air but maintain the signal from the film and fibers within the sample image. Film and/or fiber containing voxels are referred to as “material” voxels.

A connected components algorithm is executed on the trimmed 3D image, which identifies and groups together any material voxels that are 26-connected (touching one of their faces, edges, or corners) to any neighboring material voxels. Any material voxel clusters containing fewer than 1000 connected voxels are identified as noise and removed from the 3D image.

The 3D image is oriented so that the film upper surface is as close to parallel with the XY-plane as possible.

The film layer is identified and distinguished from nonwoven fibers using a Z-direction vector, such that given an XY-plane position, a typical Z-direction vector traveling from the top of the 3D image to the bottom will first pass through the film, and then pass through underlying nonwoven fibers. However, in the regions where apertures formed in the film layer, a fiber may be the first material encountered, and must be distinguished from the film layer. As an individual Z-direction vector travels from the top of the 3D image downward, there may be a series of contiguous material voxels in the vector as it passes through the first material encountered. The last material voxel in this series of contiguous material voxels is identified as a potential lower film surface or “bottom of film” voxel. This process is repeated as a Z-direction vector is passed through every XY-plane position in the 3D image, and all of the potential bottom of film voxels are identified. A connected components algorithm is once again executed on only the identified potential bottom of film voxels in the 3D image, which groups together potential bottom film voxels that are 26-connected (touching one of their faces, edges, or corners) to neighboring potential bottom of film voxels. The lower surface of the film is identified as the single largest continuous cluster of potential bottom of film voxels.

The fiber-to-fiber distance is measured along the Z-direction vectors, below the identified lower surface of the film layer from where one fiber ends to the beginning of the next underlying fiber. If no film voxel was identified in the Z-direction vector, due to a hole or aperture in the film layer, any distance measurements from that vector are ignored. Any Z-direction vectors which do not encounter any fibers are also ignored. The median fiber-to-fiber distance of all the distance measurements in the 3D image is calculated and recorded to the nearest 0.1 μm. A total of three substantially similar replicate film-nonwoven laminate samples are analyzed in like manner, and the average of the three recorded median fiber-to-fiber distances is reported to the nearest 0.1 μm.

### Combinations

In view of the foregoing disclosure, the following non-limiting examples are contemplated:
A. An absorbent personal hygiene article comprising: a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE; wherein the film layer has a basis weight of from about 14 gsm to about 24 gsm, wherein the film layer comprises a pattern of cones and valleys, the cones projecting upwardly from adjacent valleys to rim edges proximate an upper side of the film layer, wherein the cones circumscribe and define apertures through the film layer, wherein the aperture is formed at rim edge of the cone, wherein the apertures have an average largest x-y dimension (ar) of about 375 μm to about 550 μm., wherein the cones have an average height (ch) of about 300 μm to about 500 μm, wherein the apertures have a numerical density of about 140 to about 500 apertures per cm² x-y plane surface area of the film layer.
B. The absorbent personal hygiene article according to paragraph A, wherein the film layer has a basis weight of from about 16 gsm to about 20 gsm.
C. The absorbent personal hygiene according to any one of the preceding paragraphs, wherein the film layer has a basis weight of about 18 gsm.
D. The absorbent personal hygiene according to any one of the preceding paragraphs, wherein the apertures have an average largest x-y dimension (ar) of from about 400 to about 500 μm.
E. The absorbent personal hygiene according to any one of the preceding paragraphs, wherein the cones have an average height (ch) is about 400 μm.
F. The absorbent personal hygiene according to any one of the preceding paragraphs, wherein the apertures have a numerical density of 200 to 300 apertures per cm² x-y plane surface area of the film layer
G. The absorbent personal hygiene according to any one of the preceding paragraphs, wherein the apertures have a numerical density of about 220 to about 270 apertures per cm² x-y plane surface area of the film layer.
A1. An absorbent personal hygiene article comprising: a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE; wherein the film layer has a basis weight of from about 14 gsm to about 24 gsm, wherein the film layer comprises a pattern of cones and valleys, the cones projecting upwardly from adjacent valleys to rim edges proximate an upper side of the film layer, wherein the cones circumscribe and define apertures through the film layer, wherein the apertures have an average largest x-y dimension (ar) of about 375 μm to about 550 μm., wherein the cones have an average height (ch) of about 300 μm to about 500 μm, wherein the apertures have a numerical density of about 140 to about 500 apertures per cm² x-y plane surface area of the film layer; and a fluid management layer disposed directly on a lower side of the film layer, wherein the fluid management layer comprises fibers, wherein the fibers have a diameter of at least 20 microns.
A2. The absorbent personal hygiene article according to paragraph A1, wherein the fluid management layer is a carded, nonwoven.
A3. The absorbent personal hygiene according to any one of the preceding paragraphs, wherein the fluid management layer has a basis weight of from about 40 gsm to about 75 gsm.
A4. The absorbent personal hygiene according to any one of the preceding paragraphs, wherein the fibers of the fluid management layer comprise a plurality of absorbent fibers, a plurality of stiffening fibers, and a plurality of resilient fibers.
A5. The absorbent personal hygiene according to any one of the preceding paragraphs, wherein the fluid management layer has a caliper factor of at least about 0.16 mm

A6. The absorbent personal hygiene according to any one of the preceding paragraphs, wherein the fluid management layer has a caliper factor of from about 0.13 mm to about 0.3 mm.
A7. The absorbent personal hygiene article of claim A4, wherein the plurality of stiffening fibers is greater than the plurality of stiffening fibers.
A8. The absorbent personal hygiene according to any one of the preceding paragraphs, wherein the fibers of the fluid management layer have a non-circular cross section.
A9. The absorbent personal hygiene according to any one of the preceding paragraphs, wherein the fibers of the fluid management layer have a fiber to fiber distance of at least about 55 microns according to the Fiber to Fiber Distance Measurement.
A10. The absorbent personal hygiene according to any one of the preceding paragraphs, wherein the film layer is void of high density polyethylene (HDPE).
A11. The absorbent personal hygiene according to any one of the preceding paragraphs, wherein the fibers comprise bicomponent fibers, wherein the bicomponent fibers have a sheath-core configuration.
A12. The absorbent personal hygiene according to paragraph A11, wherein the sheath component comprises at least one of polyethylene and polypropylene.
B1. An absorbent personal hygiene article comprising: a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE; wherein the film layer has a basis weight of from about 14 gsm to about 24 gsm, wherein the film layer comprises a pattern of cones and valleys, the cones projecting upwardly from adjacent valleys to rim edges proximate an upper side of the film layer, wherein the cones circumscribe and define apertures through the film layer, wherein the apertures have an average largest x-y dimension (ar) of about 375 μm to about 550 μm., wherein the cones have an average height (ch) of about 300 μm to about 500 μm, wherein the apertures have a numerical density of about 140 to about 500 aperture per cm² x-y plane surface area of the film layer; and an absorbent core layer disposed directly adjacent to the topsheet, wherein the absorbent core layer comprises cellulose fibers, wherein the cellulose fibers comprise pulp fibers.
B2. The absorbent personal hygiene according to any one of the preceding paragraphs, wherein the film layer comprises no more than 5% by weight high density polyethylene, polypropylene. or polyethylene terephthalate.
B3. The absorbent personal hygiene according to any one of the preceding paragraphs, wherein the film layer comprises a hydrophilizing agent.
B4. The absorbent personal hygiene according to any one of the preceding paragraphs, wherein the film layer is bonded to the absorbent core layer.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments contemplated herein have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent personal hygiene article comprising:
a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE;
wherein the film layer has a basis weight of from about 14 gsm to about 24 gsm,
wherein the film layer comprises a pattern of cones and valleys, the cones projecting upwardly from adjacent valleys to rim edges proximate an upper side of the film layer, wherein the cones circumscribe and define apertures through the film layer, wherein the aperture is formed at rim edge of the cone, wherein the apertures have an average largest x-y dimension (ar) of about 375 μm to about 550 μm, wherein the cones have an average height (ch) of about 300 μm to about 500 μm, wherein the apertures have a numerical density of about 140 to about 500 apertures per cm² x-y plane surface area of the film layer.

2. The absorbent personal hygiene article of claim 1, wherein the film layer has a basis weight of from about 16 gsm to about 20 gsm.

3. The absorbent personal hygiene article of claim 1, wherein the film layer has a basis weight of about 18 gsm.

4. The absorbent personal hygiene article of claim 1, wherein the apertures have an average largest x-y dimension (ar) of from about 400 to about 500 μm.

5. The absorbent personal hygiene article of claim 1, wherein the cones have an average height (ch) is about 400 μm.

6. The absorbent personal hygiene article of claim 1, wherein the apertures have a numerical density of 200 to 300 apertures per cm² x-y plane surface area of the film layer.

7. The absorbent personal hygiene article of claim 1, wherein the apertures have a numerical density of about 220 to about 270 apertures per cm² x-y plane surface area of the film layer.

8. An absorbent personal hygiene article comprising:
a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE;
wherein the film layer has a basis weight of from about 14 gsm to about 24 gsm,
wherein the film layer comprises a pattern of cones and valleys, the cones projecting upwardly from adjacent valleys to rim edges proximate an upper side of the film layer, wherein the cones circumscribe and define apertures through the film layer, wherein the apertures have an average largest x-y dimension (ar) of about 375 μm to about 550 μm, wherein the cones have an average height (ch) of about 300 μm to about 500 μm, wherein the apertures have a numerical density of about 140 to about 500 apertures per cm² x-y plane surface area of the film layer; and
a fluid management layer disposed directly on a lower side of the film layer, wherein the fluid management layer comprises fibers, wherein the fibers have a diameter of at least 20 microns.

9. The absorbent personal hygiene article of claim 8, wherein the fluid management layer is a carded, nonwoven.

10. The absorbent personal hygiene article of claim 8, wherein the fluid management layer has a basis weight of from about 40 gsm to about 75 gsm.

11. The absorbent personal hygiene article of claim 8, wherein the fibers of the fluid management layer comprise a plurality of absorbent fibers, a plurality of stiffening fibers, and a plurality of resilient fibers.

12. The absorbent personal hygiene article of claim 8, wherein the fluid management layer has a caliper factor of at least about 0.16 mm.

13. The absorbent personal hygiene article of claim 8, wherein the fluid management layer has a caliper factor of from about 0.13 mm to about 0.3 mm.

14. The absorbent personal hygiene article of claim 11, wherein the plurality of stiffening fibers is greater than the plurality of stiffening fibers.

15. The absorbent personal hygiene article of claim 8, wherein the fibers of the fluid management layer have a non-circular cross section.

16. The absorbent personal hygiene article of claim 8, wherein the fibers of the fluid management layer have a fiber to fiber distance of at least about 55 microns according to the Fiber to Fiber Distance Measurement.

17. The absorbent personal hygiene article of claim 8, wherein the film layer is void of high density polyethylene (HDPE).

18. The absorbent personal hygiene article of claim 8, wherein the fibers comprise bicomponent fibers, wherein the bicomponent fibers have a sheath-core configuration.

19. The absorbent personal hygiene article of claim 18, wherein the sheath component comprises at least one of polyethylene and polypropylene.

20. An absorbent personal hygiene article comprising:
a topsheet, the topsheet comprising a film layer, wherein the film layer comprises polyethylene (PE), wherein the polyethylene is a homogeneous blend of low density polyethylene (LDPE) and linear low-density polyethylene (LLDPE), wherein the homogeneous blend is at least 50% by weight LDPE, and LLDPE;
wherein the film layer has a basis weight of from about 14 gsm to about 24 gsm,
wherein the film layer comprises a pattern of cones and valleys, the cones projecting upwardly from adjacent valleys to rim edges proximate an upper side of the film layer, wherein the cones circumscribe and define apertures through the film layer, wherein the apertures have an average largest x-y dimension (ar) of about 375 μm to about 550 μm., wherein the cones have an average height (ch) of about 300 μm to about 500 μm, wherein the apertures have a numerical density of about 140 to about 500 aperture per cm² x-y plane surface area of the film layer; and
an absorbent core layer disposed directly adjacent to the topsheet, wherein the absorbent core layer comprises cellulose fibers, wherein the cellulose fibers comprise pulp fibers.

21. The absorbent personal hygiene article of claim 20, wherein the film layer comprises no more than 5% by weight high density polyethylene, polypropylene, or polyethylene terephthalate.

22. The absorbent personal hygiene article of claim 20, wherein the film layer comprises a hydrophilizing agent.

23. The absorbent personal hygiene article of claim 20, wherein the film layer is bonded to the absorbent core layer.
